(19) United States
(12) Patent Application Publication
(10) Pub. No.: US 2025/0157238 A1
(43) Pub. Date: May 15, 2025

(54) COMPUTER IMPLEMENTED METHOD OF EXTRACTING DATA FROM SURVEYS

(71) Applicant: Research Grid Ltd, London (GB)

(72) Inventors: Amber Michelle Hill, London (GB); Tushar Joshi, London (GB)

(21) Appl. No.: 18/506,678

(22) Filed: Nov. 10, 2023

(51) Int. Cl. G06V 30/148; G06F 16/93; G06V 30/412; G16H 10/20

(52) U.S. Cl. CPC G06V 30/148; G06F 16/93; G06V 30/412; G16H 10/20

(57) ABSTRACT

A computer-implemented method of extracting data, such as clinical patient information from surveys such as medical surveys is disclosed herein. The method comprises obtaining completed surveys, each completed survey comprising answers to preconfigured questions, identifying portions of the survey corresponding to answers, applying a machine learning model to the portions identified as answers to predict answers, and accumulating a response to the survey based on the predicted answers.

### FIELD OF THE DISCLOSURE

The present disclosure relates to a computer-implemented method of extracting data, such as clinical patient information, from surveys, such as medical surveys.

### BACKGROUND

Surveys or assessments are often conducted either via paper (hard copy) or electronically (soft copy) and are often used to obtain information from a group of selected individuals. For example, during a medical study, clinical trial, or participant engagement exercise, researchers or hosts are required to collect responses from participants (which may be patients in the context of a medical survey), for example to establish feedback from an activity, such as the effectiveness of a drug. These responses may be collected via a survey. This process is time consuming and extensively manual driven. For example, to upload and convert the data of one survey from a paper-based form to an electronic format where it can be more easily processed, may take the host or researcher around 45 min to 8 hours.

Embodiments of the disclosure seek to address these problems.

### SUMMARY OF THE DISCLOSURE

Aspects of the invention are as set out in the independent claims and optional features are set out in the dependent claims. Aspects of the invention may be provided in conjunction with each other and features of one aspect may be applied to other aspects.

Embodiments of the disclosure provide a system which can be used to upload scanned copies of medical surveys (in particular, images of the medical surveys), enabling extracted questions and their dependent responses to be obtained and accumulated. The data can then be structured into a json/xml/csv data type and uploaded into a system where the researchers are running their study. This service can also be provided as an endpoint to other players in the market. The expectation is that this “intelligent automation” can be used to bring down process time from hours to seconds.

Accordingly, in a first aspect there is provided a computer-implemented method of extracting data, such as clinical patient information, from a survey (sometimes referred to as assessments or questionnaires), such as a medical survey. Clinical patient information may comprise data such as specific responses to specific questions, for example regarding their current state of health. The method comprises obtaining completed surveys, each completed survey comprising answers to preconfigured questions, identifying portions of the survey corresponding to answers, applying a machine learning model to the portions identified as answers to predict answers, and accumulating a response to the survey based on the predicted answers. It will be understood that identifying portions of the survey as corresponding to answers may comprise making a prediction that the portion corresponds to answers. Applying a machine learning model to the portions identified as answers to predict answers may comprise performing computer vision, such as image recognition, on an image of the survey. For example, image classification and/or image segmentation may be performed.

Accumulating a response to the survey based on the identified answers may comprise matching the identified answers to the preconfigured questions. The preconfigured questions may be preconfigured questions for a preconfigured survey corresponding to the completed survey.

The method may further comprise outputting a display of the answers to each question in a graphical user interface to the user, along with an indication of the location of the answer in the survey and the confidence level that the answer has been correctly predicted by the second machine learning model. In some examples, the user may be presented, for example via a graphical user interface, with a view of the survey with an overlay of the identified questions/answers. This enables the user to see the questions and answers as identified by the machine learning model. In some examples, the machine learning model may identify the questions and answers (or question and answer sections) by placing boxes around them. In some examples, these boxes may be adjusted by the user via the graphical user interface. A question and answer section may comprise one or more question portions and one or more answer portions.

In some examples, the machine learning model may be configured to automatically determine and flag any errors to the user, for example via the graphical user interface. For example, if the confidence level of a predicted answer is less than a selected threshold level, this will be indicated on the graphical user interface to the user. Additionally, or alternatively, if the machine learning model is unable to identify a question or answer section, this may also be flagged to the user—for example if a question section is identified but a corresponding answer section is not identified, this may be flagged to the user.

In some examples, the method may comprise classifying sections of the survey as question and answer sections. Identifying portions of the survey corresponding to answers may comprise identifying a portion corresponding to one or more answers for each classified section of the medical survey. There may be a plurality of answer portions for each classified question and answer section. There may also be a plurality of question portions for each classified question and answer section. For example, each question and answer section may comprise an overarching question and then sub-question portions with answer portions for each sub-question portion.

In some examples, the method comprises classifying the question and answer sections of the survey into one of a selected number of types. This may comprise applying a machine learning model, for example an image classification model such as a convolutional neural network to perform image segmentation. The question and answer sections may comprise one or more types selected from: I block, single select QnA block, multi-select QnA block, table block, scale select, and body part image mark. The machine learning model may be configured to predict the answer for each type based on the identified type of question and answer section.

Applying a machine learning model to the portions identified as answers to predict answers may comprise applying a machine learning model to the portions identified as answers to predict answers for each classified section based on the classified type of the section. In other words, the information on the classification of each section (for example, whether it is a question or answer section, and/or what type of answer it is) may be fed as an input to the machine learning model. The machine learning model may be a single shot detection (SSD) model, for example available from PyTorch®.

As noted above, it may be preferable to use images of the surveys so that machine learning models trained in computer vision may be used. Accordingly, in some examples obtaining completed surveys comprises obtaining an image of completed surveys. This may comprise converting a PDF of the survey to an image.

In some examples the method further comprises obtaining, from a user, an indication from a list of preconfigured surveys of the survey being obtained.

In some examples the method further comprises determining whether each of the obtained completed surveys belongs to a list of preconfigured surveys; and in the event that the obtained completed survey is determined to belong to the list of preconfigured medical surveys, suggesting to the user a recommended survey to select from the list of preconfigured medical surveys; and in the event that the obtained completed medical survey does not belong to the list of preconfigured surveys, prompting the user to provide a blank copy of the medical survey. For example, the method may comprise a machine learning model configured to perform text similarity matching. This may involve using an OCR product, such as GCP® Vision or AWS® Textract.

As well as identifying portions of each completed survey corresponding to answers, the method may also comprise identifying portions of the survey corresponding to questions. This may be performed when identifying question and answer sections of the survey, and may be performed before portions of each completed medical survey corresponding to answers are identified.

Identifying portions of the medical survey corresponding to questions may comprise applying (i) a layout machine learning model configured to split the image into portions that are questions, and (ii) an extract machine learning model configured to extract a question from each portion.

Similarly, in some examples, identifying portions of the obtaining completed survey corresponding to answers comprises applying (i) a layout machine learning model configured to split the image into portions that are answers, and (ii) an extract machine learning model configured to extract an answer from each portion.

In some examples, identifying portions of the medical survey corresponding to answers comprises generating boxes with size ratio and variable size around portions of the medical survey and then matching the identified portions to preconfigured portions of a preconfigured survey. This may comprise applying a machine learning model, for example a Structural similarity index measure (SSIM). Instead of determining direct similarity this may use a combination of vision and text to calculate the similarity—true images should have more similarities than a false image, (similarity between pg1. matching pg. 1 uploads (resizing images, changing interpolation concepts).

In another aspect of the disclosure, there is provided a computer-implemented method of extracting data, such as clinical patient information, from surveys such as medical surveys (sometimes referred to as assessments or questionnaires). The method comprises obtaining completed surveys, each completed survey comprising answers to preconfigured questions, identifying portions of the survey corresponding to questions, and identifying portions corresponding to answers, to find the closest match with a corresponding portion in the preconfigured survey, and accumulating a response to the survey for each identified portion of the medical survey using the identified questions and identified answers by predicting a user selection using an object detection machine learning algorithm.

The method may comprise, for each page of the obtained completed survey, identifying the page of a preconfigured survey to which the page corresponds.

The method may comprise extracting question and answer information from the identified portions of the survey to accumulate the responses to the survey.

The method may comprise extracting Principal Investigator, PI, information from the survey and anonymizing the PI data.

Identifying portions of the survey corresponding to questions, and identifying portions corresponding to answers, to find the closest match with a corresponding portion in the preconfigured medical survey, may comprise applying a machine learning model to generate boxes with size ratio and variable size and using a neural network to find the closest match with a corresponding portion in the preconfigured survey This may comprise applying a machine learning model, for example a Structural similarity index measure (SSIM). Instead of determining direct similarity this may use a combination of vision and text to calculate the similarity-true images should have more similarities than a false image, (similarity between pg1. matching pg. 1 uploads (resizing images, changing interpolation concepts).

In some examples, the method comprises the optional step of identifying question and answer sections based on the identified question and answer portions. Additionally, or alternatively, the question and answer portions may be identified based on an identification of question and answer sections. A question and answer section may comprise one or more question portions and one or more answer portions.

In some examples, the method comprises classifying the identified question and answer portions, or question and answer sections, of the medical survey into one of a selected number of types. This may comprise applying a machine learning model, for example an image classification model such as a convolutional neural network to perform image segmentation. The question and answer sections may comprise one or more types selected from: I block, single select QnA block, multi-select QnA block, table block, scale select, and body part image mark. The machine learning model may be configured to predict the answer for each type based on the identified type of question and answer section. In such examples, accumulating a response to the medical survey may be based on the classified type.

The method may further comprise outputting a display of the answers to each question in a graphical user interface to the user, along with an indication of the location of the answer in the survey and the confidence level that the answer has been correctly predicted by the second machine learning model.

In some examples, the user may be presented, for example via a graphical user interface, with a view of the medical survey with an overlay of the identified question/answers. This enables the user to see the questions and answers as identified by the machine learning model. In some examples, the machine learning model may identify the questions and answers (or question and answer sections) by placing boxes around them. In some examples, these boxes may be adjusted by the user via the graphical user interface. A question and answer section may comprise one or more question portions and one or more answer portions.

In some examples, the machine learning model may be configured to automatically determine and flag any errors to the user, for example via the graphical user interface. For example, if the confidence level of a predicted answer is less than a selected threshold level, this will be indicated on the graphical user interface to the user. Additionally, or alternatively, if the machine learning model is unable to identify a question or answer section, this may also be flagged to the user—for example if a question section is identified but a corresponding answer section is not identified, this may be flagged to the user.

In some examples, the machine learning model may be configured to automatically determine and flag any errors to the user, for example via the graphical user interface. For example, if the confidence level of a predicted answer is less than a selected threshold level, this will be indicated on the graphical user interface to the user. Additionally, or alternatively, if the machine learning model is unable to identify a question or answer section, this may also be flagged to the user—for example if a question section is identified but a corresponding answer section is not identified, this may be flagged to the user.

In another aspect there is provided a computer-implemented method of extracting information, such as clinical patient information, from surveys, such as medical surveys. The method comprises obtaining completed surveys, each completed survey comprising answers to preconfigured questions, applying a question machine learning model to identify and predict instances of questions in each completed survey, applying a separate answer machine learning model to identify and predict answers in each completed survey, and accumulating a response to the survey based on the predicted answers. Advantageously this may only require two machine learning models to be trained: a question model and an answer model, which may be more efficient.

Obtaining completed surveys may comprise obtaining an image of each completed survey.

Applying a question machine learning model to identify and predict instances of questions in each completed survey may comprise the steps of (i) splitting the image into portions that are questions, and (ii) extracting a question from each portion.

Extracting a question may comprise providing a prediction of an instance of a question.

Splitting the image into portions that are questions may comprise identifying portions by generating boxes with size ratio and variable size around portions of the survey and then matching the identified portions to preconfigured portions of a preconfigured medical survey. In some examples, a user may be able to correct the generated boxed/predictions of bounding boxes and the selection of the identified questions, and/or correct these manually.

Applying an answer machine learning model to identify and predict answers in each completed survey may comprise the steps of (i) splitting the image into portions that are answers, and (ii) extracting an answer from each portion. Extracting an answer may comprise providing a prediction of an answer.

Splitting the image into portions that are answers comprises identifying portions by generating boxes with size ratio and variable size around portions of the medical survey and then matching the identified portions to preconfigured portions of a preconfigured medical survey.

In another aspect there is provided a graphical user interface configured to display the results of surveys, such as medical surveys. The graphical user interface configured to, on a first page, prompt a user to upload a blank survey, on a subsequent page, provide an indication to the user of each identified question and answer section of the blank survey along with a question and answer type, and prompting the user to correct any misidentified areas or question and answer types, and on a subsequent page, provide an indication to the user of the answers to each identified question and answer section for each completed survey based on the identified question and answer type for that identified question and answer section, along with an indication of the confidence level of the indication for each completed survey and answer section for each completed medical survey.

In another aspect there is provided a computing device comprising a display screen, the computing device being configured to display on the screen: a prompt to a user to upload a blank survey, after the user has uploaded a blank survey, an indication to the user of each identified question and answer section of the blank survey along with a question and answer type, and prompting the user to correct any misidentified areas or question and answer types, a prompt to a user to upload completed surveys, and subsequently, an indication to the user of the answers to each identified question and answer section for each completed medical survey based on the identified question and answer type for that identified question and answer section, along with an indication of the confidence level of the indication for each completed survey and answer section for each completed medical survey.

In another aspect there is provided a computer readable non-transitory storage medium comprising a program for a computer configured to cause a processor to perform the method of any of the aspects described above.

### BRIEF DESCRIPTION OF THE DRAWINGS

Embodiments of the disclosure will now be described, by way of example only, with reference to the accompanying drawings, in which:

FIG. 1 shows a screenshot of an example completed medical survey;

FIG. 2 shows a first page of an example graphical user interface;

FIG. 3 shows another page of an example graphical user interface showing a blank medical survey being uploaded;

FIG. 4 shows another page of an example graphical user interface showing portions of the medical survey indicating questions being highlighted;

FIG. 5 shows another page of an example graphical user interface showing portions of the medical survey indicating questions being highlighted

FIG. 6 shows another page of an example graphical user interface where a user can upload completed medical surveys;

FIG. 7 shows another page of an example graphical user interface showing thumbnails of at least a portion of each uploaded completed medical survey;

FIG. 8 shows another page of an example graphical user interface showing an indication of each answered question along with the predicted answer and the corresponding confidence level of the identification or prediction of the answer;

FIG. 9 shows a process flow-chart of an example process flow for analyzing blank and completed medical surveys using one or more machine learning models;

FIG. 10 shows an alternative method of extracting clinical patient information from medical surveys;

FIG. 11 shows a flow chart of an example computer-implemented method of extracting clinical patient information from medical surveys;

FIG. 12 shows a flow chart of another example computer-implemented method of extracting clinical patient information from medical surveys;

FIG. 13 shows a flow chart of another example computer-implemented method of extracting clinical patient information from medical surveys;

FIG. 14 shows a process flow chart for the steps performed by a graphical user interface for displaying the result of medical surveys; and

FIG. 15 is a block diagram of a computer system suitable for implementing one or more embodiments of the present disclosure.

### DETAILED DESCRIPTION

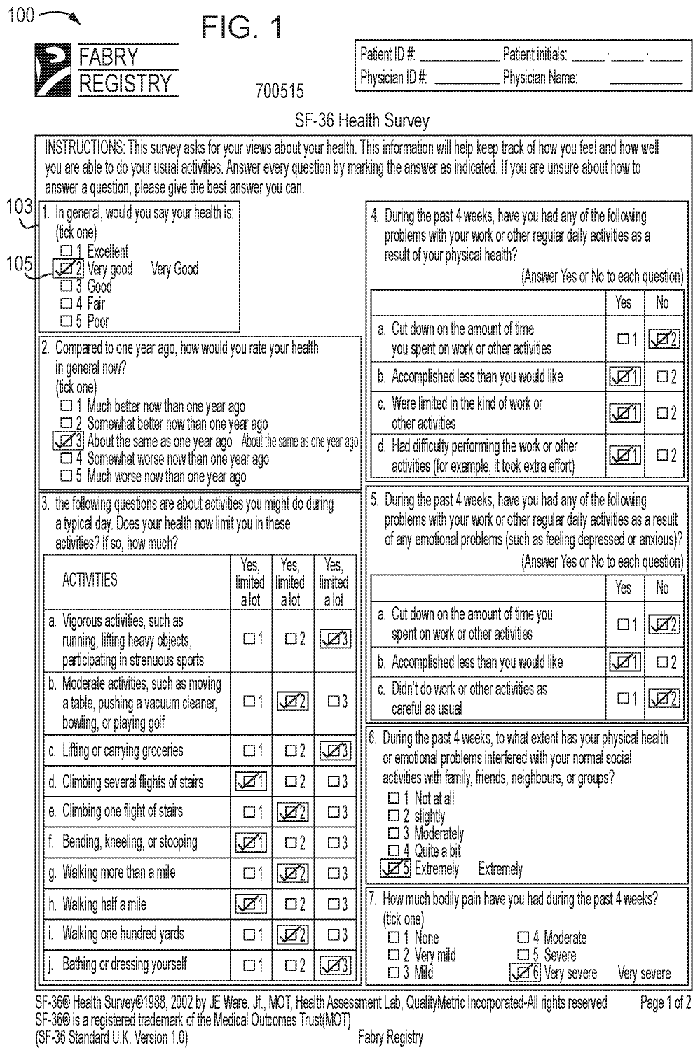

FIG. 1 is a screenshot of an example completed medical survey 100 that represents an ideal scenario that has been processed by a machine learning model. As one can see the machine learning model has performed computer vision, and in this instance image recognition, to identify sections of question and answer sections 103 and also able to identify user's selections (also referred to as answers) 105 within each question and answer section 103. Once this is achieved a response can be attached to each question to create a data dump of the patient survey response. In the present case, in FIG. 1, one can see how the machine learning model performing computer vision has identified boxes around each individual question and answer section, and also around each answer for each question section. As can be seen in FIG. 1, for some question sections there may be a plurality of answer sections. In some examples, the machine learning model may also identify the text corresponding to the answer—for example, if the answer comprises a marking adjacent to one of a number of different options, the machine learning model may identify and extract the text content of that answer adjacent to the marking made by the user (as shown in FIG. 1). Although the example given here relates to use with medical surveys, it will be appreciated that the methods described herein could be applied to a variety of different surveys or assessments of a different nature.

As shown in FIG. 1, for many of the question and answer sections, there are a plurality of answer portions. There may be a corresponding sub-question portion corresponding to each answer portion for each question and answer section.

The methods described herein may make use of three different machine learning models at three different stages. This may include a structure similarity model, an image classification model, and a single shot detection (SSD300) model. The structured similarity model may be used to extract question and answer sections from uploaded data images of the surveys/assessments. Image classification may be used to detect the marked answers from the submitted surveys and assessments. Single shot detection may be used to link the submitted answers to their relevant questions and the participants.

While with enough data and resources a single large model could be used for these tasks, when the datasets are smaller, like most cohort sizes in clinical trials, using a number of separate models enables a quicker solution of the problem to be realized. Furthermore, using a combination of different models enables various details to be extracted from complex and multi-structured surveys and assessments more accurately. In particular, the variety and structure of surveys and assessments are different and small. Each data file has several components that are unstructured and may be organized differently. Therefore a combination of models is used for different components of the surveys/assessments that need to extract specific features or information. Furthermore, the complexity of features and information from smaller cohorts of participants (e.g., patients in the context of medical surveys) is more accurately extracted using a combination of multiple models. Every model has a different specialization specific for the aforementioned features of survey and assessment data (i.e., different input and output), hence they will have different accuracy against different types of data.

The methods described herein also involve converting unstructured data (i.e., completed surveys) into structured data. This may involve a transformation into a json or xml format, for example. The data may be multi-hierarchical, hence tabular results would not establish the multi-level relationships. The unsorted data is transformed into sorted data (for example, via the machine learning models described above) where key value pairing transforms it from unstructured data into structured data.

The inventors have found that due to there being a limitation in the number of medical surveys available for training a machine learning model, a single model would not be suitable unless there are images in thousands (50K +) which at-least cater to 5K surveys. The inventors have therefore found that the task of interpreting medical surveys can be achieved more efficiently by breaking it into smaller achievable steps. The inventors have found that the use of pre-initiated information can advantageously minimize error. The inventors also propose reporting the confidence of the predictions to users.

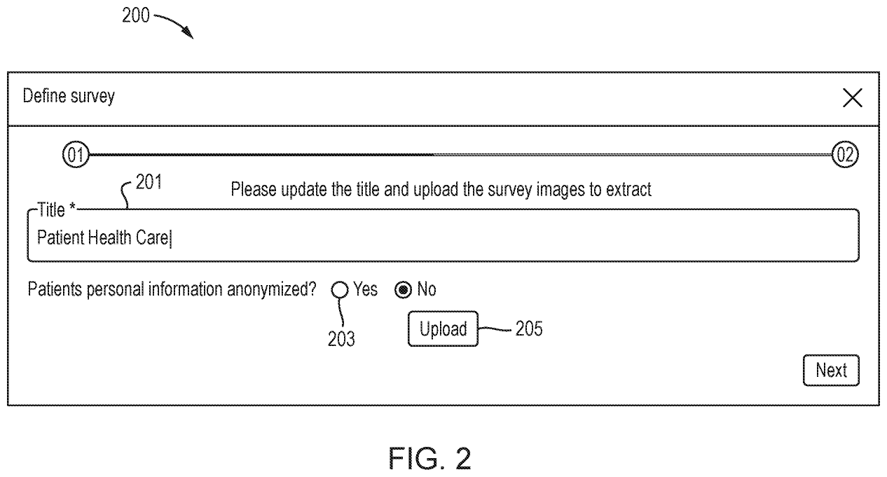

FIGS. 2 to 8 show a graphical user interface configured to display the results of medical surveys. FIG. 2 shows on a first page 200 of the graphical user interface a prompt to a user. The prompt asks a user to indicate the title 201 for the survey. The user is then asked via radio buttons 203 to select whether the patient personal information is anonymized or not. There is also a button 205 prompting the user to upload a blank medical survey. Clicking on “upload” button 205 enables the user to select a blank (i.e., not completed) medical survey to be uploaded. Uploading a blank survey may comprise transforming the survey into an image format (such as jpg) so that computer vision techniques can be applied to it.

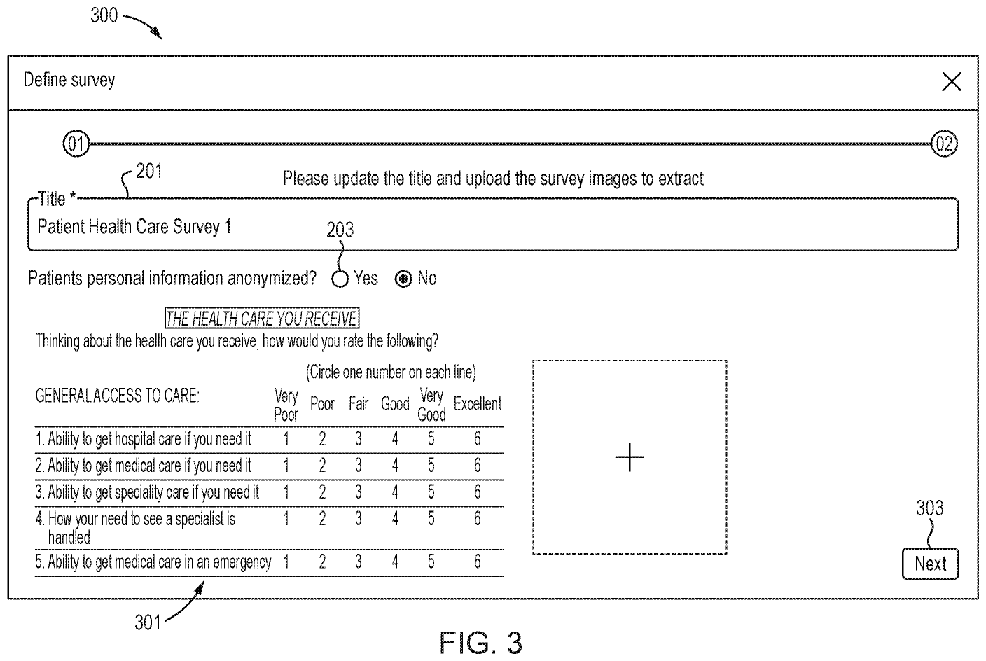

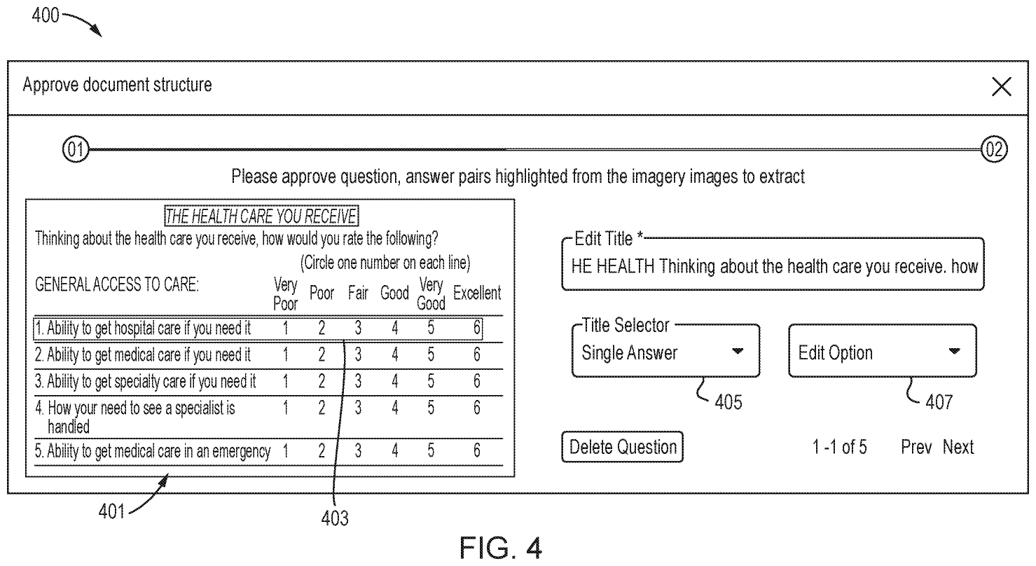

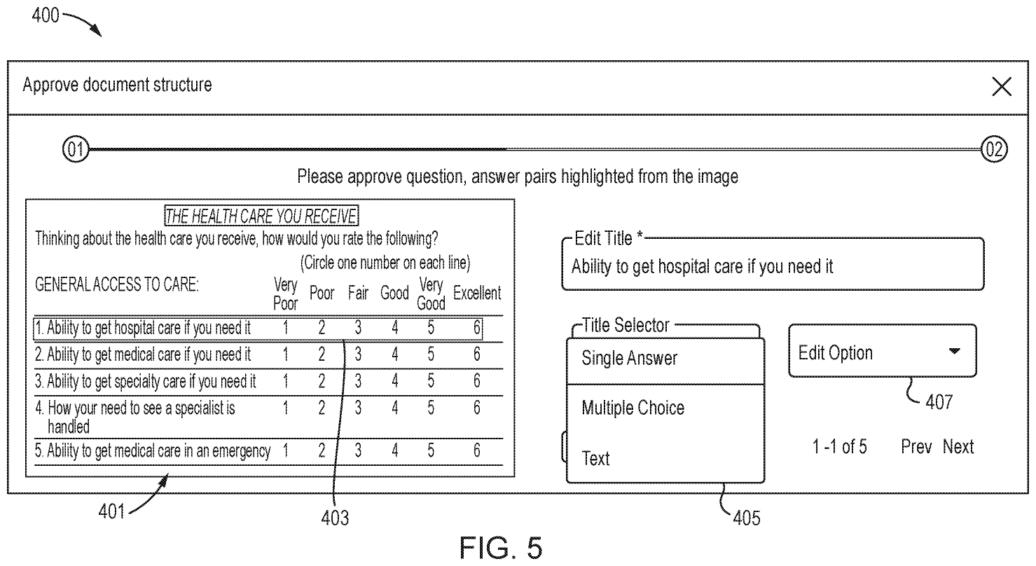

Once the user has performed this, the user may be presented with a page 300 as shown in FIG. 3. As can be seen in FIG. 3, a blank medical survey, or a portion thereof, has been uploaded and a thumbnail 301 of that medical survey is presented to the user. If the user clicks on the “next” button 303, they may then be taken to the page 400 as shown in FIG. 4.

As shown on page 400 of FIG. 4, the user is presented with a highlighted portion 403 of a thumbnail view 401 of the medical survey. The highlighted portion has been identified by a machine learning model analysing the uploaded blank medical survey as being a question/answer section. Alongside this, the user is prompted to select the answer type (e.g., single answer) 405 and also an “edit option” box 407 for editing the answer type. For example, as shown in FIG. 5, the user can select between “single answer”, “multiple choice” or “text” as the answer type.

These prompts allow the user to predefine or preconfigure the medical survey, so that when completed medical surveys are later uploaded, they can be compared to the predefined or preconfigured medical survey (for example, by comparing questions of the completed medical survey with predefined or preconfigured questions), which may improve accuracy of identification of both questions and user selected answers to those questions.

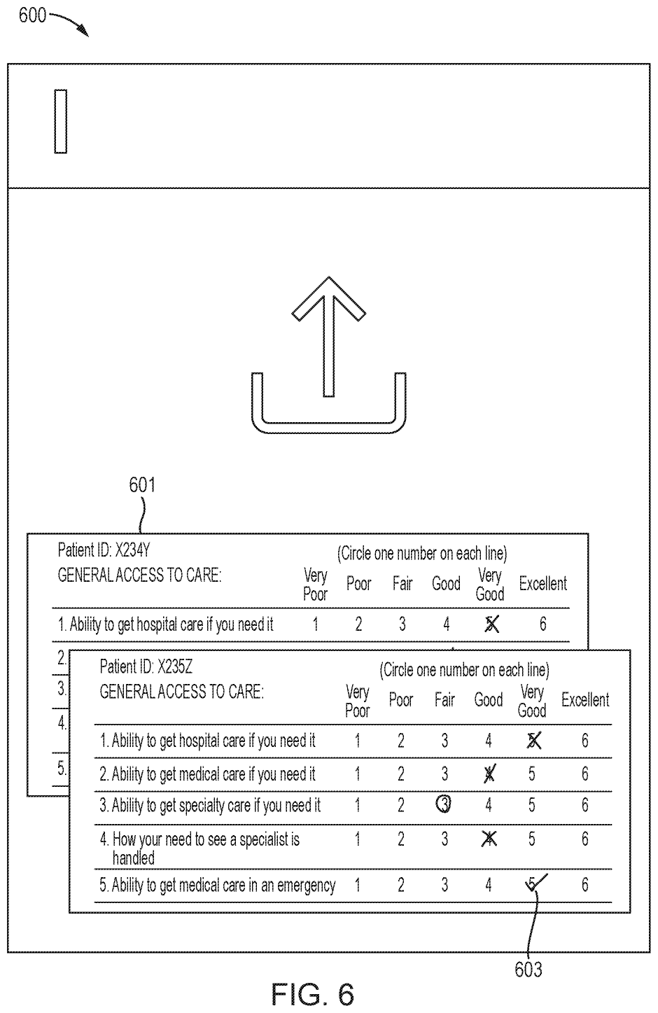

Once the user has completed the above for all the identified question/answer sections of the blank medical survey, they may then upload a blank medical survey, as shown for example, in FIG. 6. As shown in FIG. 6, the user may be prompted to upload a plurality of completed medical surveys 601 on page 600. Each completed medical survey 601 may include hand annotated answers 603 (for example, markings such as ticks, crosses or circles).

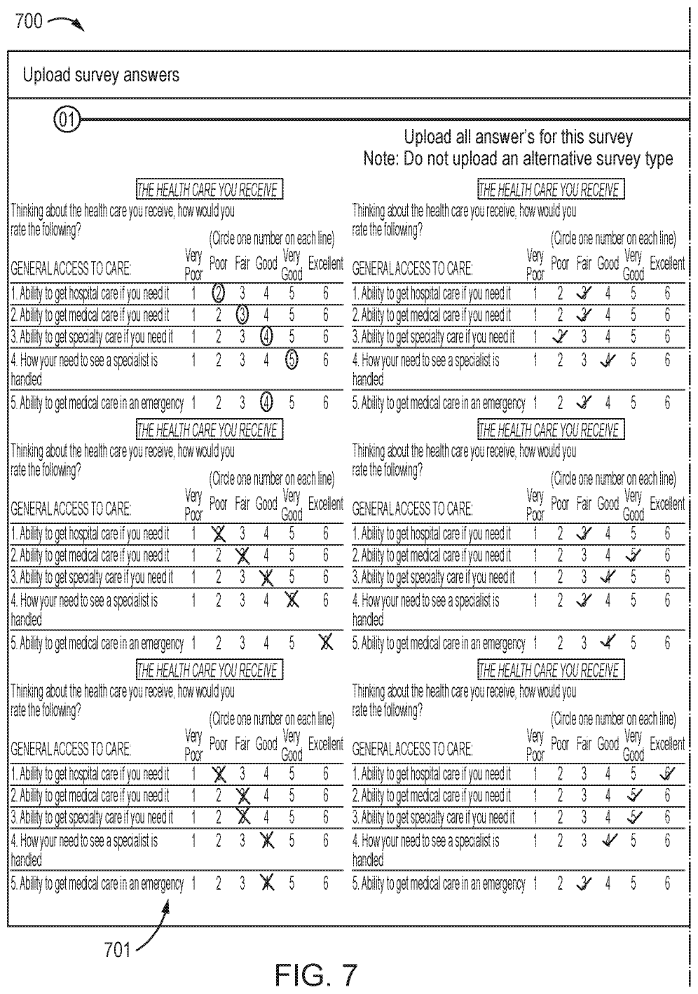

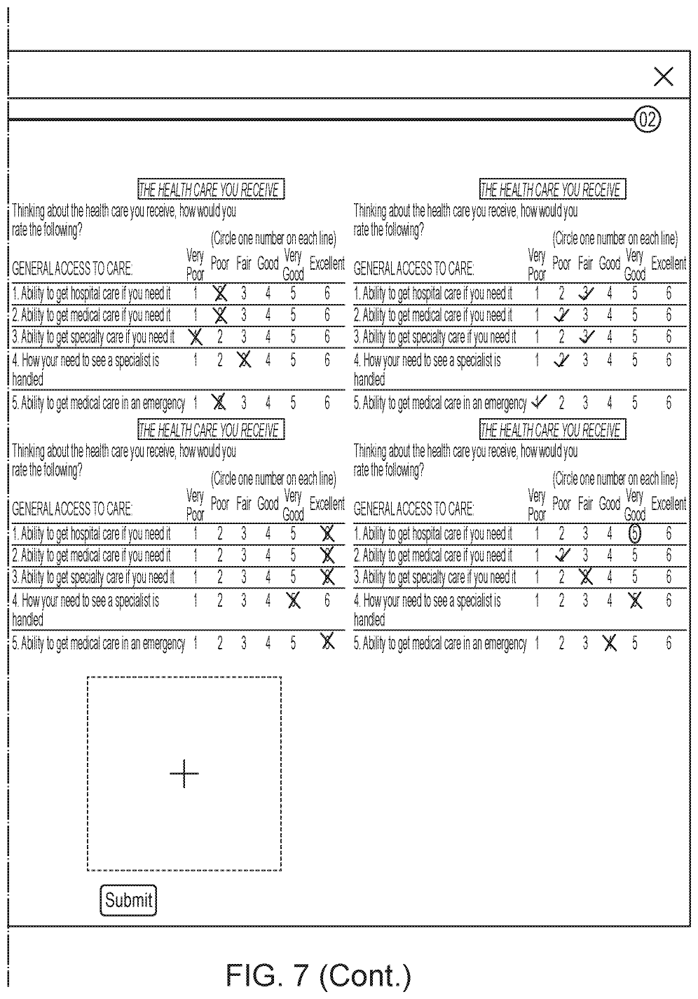

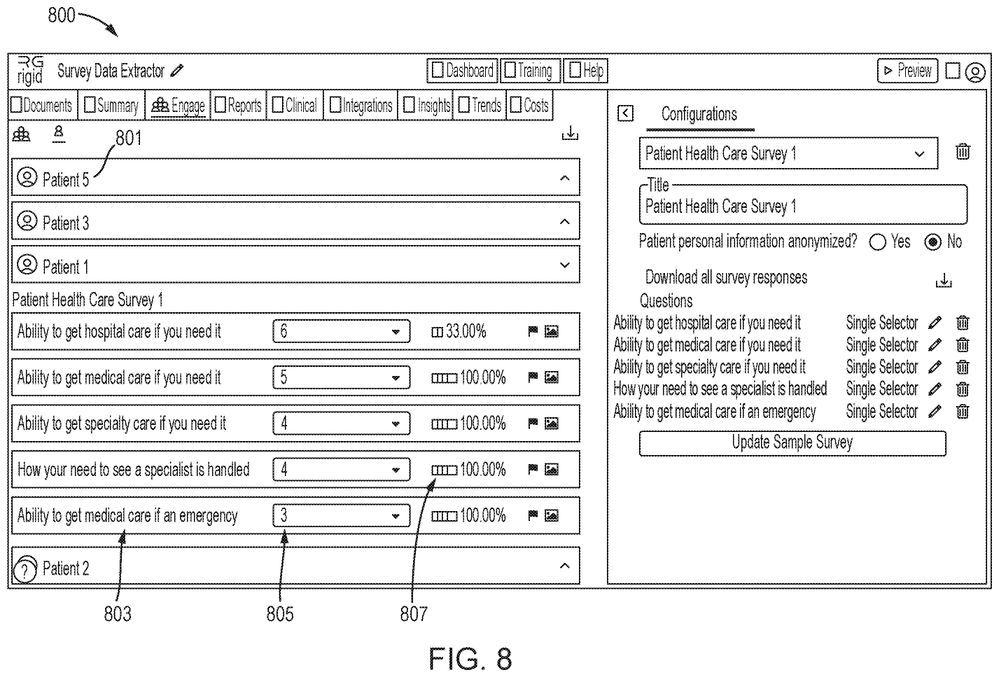

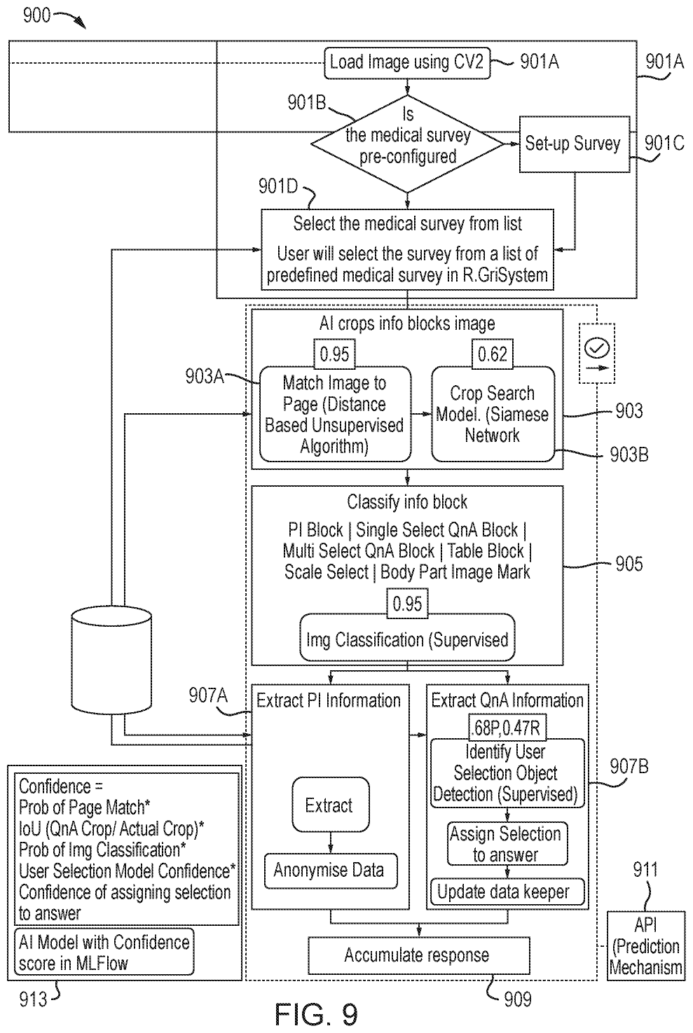

Once this is done, as shown in FIG. 7, the user is then presented with another page 700 showing thumbnails of at least a portion 701 of each uploaded completed medical survey. The completed medical surveys are then analysed using a technique that is described in more detail below with reference to FIGS. 9 to 14, before the user is presented with another page 800, as shown in FIG. 8. As can be seen in FIG. 8, this page 800 presents the user, for each identified patient 801, with an indication of each answered question 803 along with the predicted answer 805 and the corresponding confidence level 807 of the identification or prediction of the answer. It is to be understood that the indication of the answer is merely a prediction of the answer as determined by a machine learning model, and sometimes the prediction made by the machine learning model may not always be correct. Providing an indication of the confidence level of the prediction enables a user, when reviewing the identified answers of the completed medical surveys, to easily spot or identify any instances where the machine learning model may have been correct. For example, a flag or other indication may be provided to the user to review the survey answers, for example manually through visual inspection, if the confidence level is below a selected threshold.

The computer implemented method used to process the medical surveys will now be described in more detail below with reference to FIGS. 9 to 14.

FIG. 9 shows a process flow-chart of an example process flow 900 for analysing the blank and completed medical surveys using one or more machine learning models. A number of steps are shown in FIG. 9. At step 901 completed medical surveys are obtained by being uploaded by a user to the system. In particular, step 901 may comprise the steps of loading images 901A (rather than, for example, a pdf document) of the completed medical surveys using, for example, CV2, checking at step 901B whether the medical survey is preconfigured, and either at step 901C preconfiguring a blank survey (for example, according to the method described above with reference to FIGS. 2 to 5) or at step 901D selecting the medical survey from a list of preconfigured surveys.

It is noted that in some examples, however, this may be automated to suggest the survey to the user using similarity matching (for example, text similarity matching using an OCR product as discussed above). This may comprise applying a machine learning model, for example a Structural similarity index measure (SSIM). Instead of determining direct similarity this may use a combination of vision and text to calculate the similarity-true images should have more similarities than a false image, (similarity between pg1. matching pg. 1 uploads (resizing images, changing interpolation concepts).

An image of the survey (for example, in .jpg format) is preferable because machine learning models that have been configured for image segmentation may be used. If images of the surveys are not immediately available, the method may comprise converting the uploaded surveys (for example, in pdf format) to an image format (such as jpg).

An example of image segmentation performed on an image of a medical survey is shown in FIG. 1. As can be seen in FIG. 1, there are boxes formed around each question of the survey and each answer for each question respectively.

When blank surveys are uploaded, a recording is effectively made of the Question and Answer (“QnA”) section on the form image and its possible options. A section may be a crop of an image. This will help later to ensure that data converted into json format does not have any errors.

The user would then select 901D their respective survey from the list of surveys.

The next step involves identifying at step 903 portions of the medical survey corresponding to questions and answers. This may comprise identifying the QnA set of the preconfigured survey to which the QnA set of the completed medical survey belongs. The step of identifying at 903 portions of the medical survey corresponding to questions and answers may involve using a machine learning model to crop information blocks from the image of the completed medical surveys. Every survey (both blank and completed) may contain multiple pages. In the example shown in FIG. 9, the model may be configured to be able to identify the right page so that the correct QnA set may be aligned. In other words, this means that the model can determine which page of a completed medical survey corresponds to which page of a preconfigured survey, such that each question of the completed medical survey can be mapped to a preconfigured question of the preconfigured survey.

This is shown as step 903A in FIG. 9. In examples, the model may use text similarity matching to compare pages to determine the correct aligned page.

Once the page is identified the QnA section needs to be identified. This is shown as step 903B in FIG. 9. This may be done using a machine learning model such as a siamese network. For example, a machine learning model or algorithm may generate boxes with size ratio and variable size, and a siamese network is used to find the closest match. For example, the closest match may be a corresponding box with the closest similarity in terms of dimensions. However, it will be appreciated that other machine learning models may be used, such as a structured similarity model, Image classification and Single shot detection.

Every extracted section box may then be classified 905 into its type. This is because the type of the question and answer or QnA section may affect how user selections (i.e., answers) are extracted. For example, the method of extraction may vary based on the type. For example, the type of each QnA section may be any one of: Option, Table, Scale, Image, Text. The information on the classification of each section (for example, whether it is a question or answer section, and/or what type of answer it is) may be fed as an input to the machine learning model. The machine learning model may be a single shot detection (SSD) model, for example available from SSD300.

The user's selection may then be identified. In other words, a prediction of the answers (user's selection) is made 907B using a machine learning model and confidence ratings model 913 as indicated in FIG. 9. This may involve supervised object detection. For example, the same model as used for question identification may be used. The model may identify the location of the answers in the survey, and then identify the value (i.e., answer) given by the user. The output of this may be a prediction of the correct answer. A confidence level or indicator of the degree of confidence the model has in the prediction may also be output, for later use by a researcher/user when reviewing the extracted medical surveys. Outputs of the machine learning model may include the confidence of the prediction, the probability of the page match, Intersection over Union (IoU), probability of image classification, user selection model confidence, and confidence of assigning selection to answer.

In the example shown in FIG. 9, the same machine learning model 913 may be used to perform steps 901, 903, 905 and 907, however, it will be appreciated that in other examples each step may involve the use of a different machine learning model trained specifically for its purpose.

In some examples the model is configured to extract 907A the Principal Investigator, PI, information which may be attached on the form. But due to access restriction that data cannot be shared. The PI information may be handwritten which would be difficult to extract (error prone). In some examples, the PI information may be later completed/entered into the system by the researcher after the extraction to avoid errors. If any PI information is extracted, it will be anonymized.

Once a user's selection (i.e., the answers) is captured its attached option can be found by backtracking overlap of the selection box with the original box. The attached option may be the value or classification assigned by the machine learning model, or user, to that question/answer structure during training. For example, during training of a new survey or assessment, the machine will pick up a question with answers (i.e. ‘an option’) and it will ‘assign’ (i.e. sets the answer for that question) to either single or multiple choice, etc. It further asks the user if this ‘assignment’ for that question option is correct. So, “attached option” means the expected answer format for that question.

At step 909 a response to the medical survey is accumulated based on the predicted answers. This may involve collating the predictions with the greatest level of confidence for each QnA section for each completed medical survey. Accumulating a response to the medical survey may comprise consolidating the responses/user selection to each Q&A section of each medical survey to provide an indication of the answers to each entire medical survey.

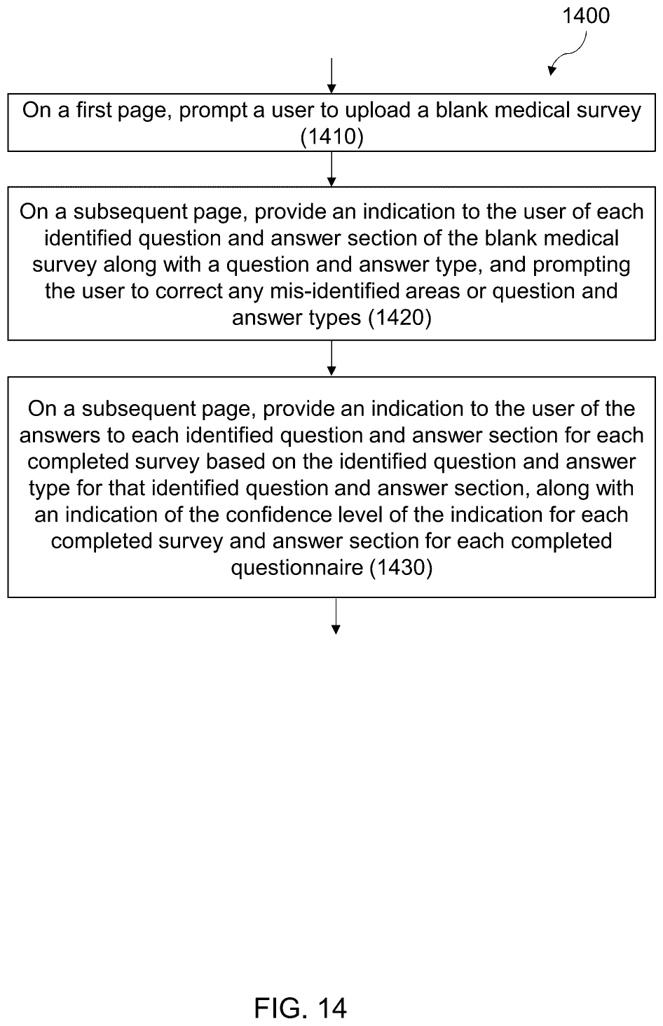

In some examples, as shown in FIG. 9, an API 911 may then be used to communicate with a program running a graphical user interface. The API 911 may be configured to receive the accumulated response at 909 and output it via a display to a user, as will be described in more detail below with reference to, for example, FIG. 14.

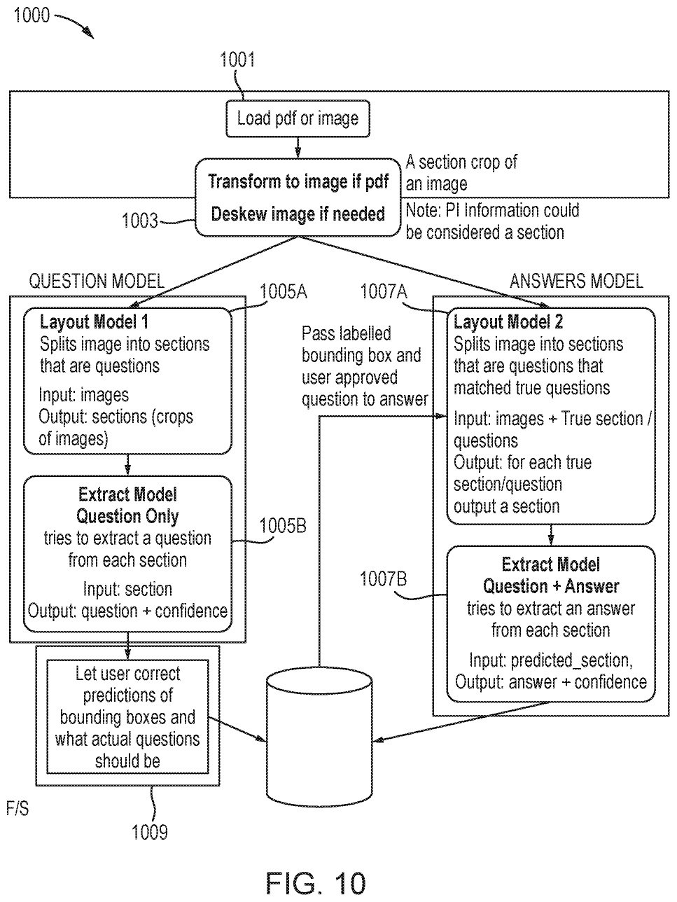

FIG. 10 shows an alternative method of extracting clinical patient information from medical surveys. Unlike the method of FIG. 9 described above, the method shown in FIG. 10 involves two separately trained machine learning models, a “question model” and an “answer model”.

Like the method describes above with reference to FIG. 9, in FIG. 10 the method begins by obtaining at 1001 copies of the completed medical surveys. If the completed medical surveys are in pdf format, at step 1003 the completed medical surveys may be converted or transformed into an image format, and the image de-skewed if necessary.

Although not shown in FIG. 10, at this point, the steps of determining whether the medical survey is preconfigured and either preconfiguring a blank survey or selecting the medical survey from a list, may be performed, although this may not be required because a question model is used, as described in more detail below.

Two processes may then be performed, for example in parallel or in series. This may involve a question model and an answer model. It will be understood that each question model and each answer model may comprise respective layout models and extract models.

At step 1005A, a question model (for example, a first layout model) is used to split the image into sections that are questions and using images as input output sections (crops of images). At step 1005B the model (for example, a first extract model) is used to extract a question from each section using a section as input and outputting an indication of the question with a confidence level. The output of this may include an indication of the bounding boxes used to perform the cropping or section selection, such that a user can modify the section selection, for example by moving or adjusting the bounding box. In this way, at step 1009, a user may correct predictions of bounding boxes and what actual questions should be.

At step 1007A, which may be performed in parallel or subsequent to steps 1005A and 1005B, an answers model (for example, a second layout model) may be used to split the image into sections that matched true questions. A true question may be the question extracted from the original image (i.e., the blank infilled survey, without answers). This may involve taking as input the image of the medical survey with a true section/questions (i.e., the question identified at step 1005B), and output a section for each true section/question. At step 1007B, the model (for example, a second extract model) may extract an answer from each section. It may take as an input a prediction of a section and output an answer to that section along with a confidence level of that prediction.

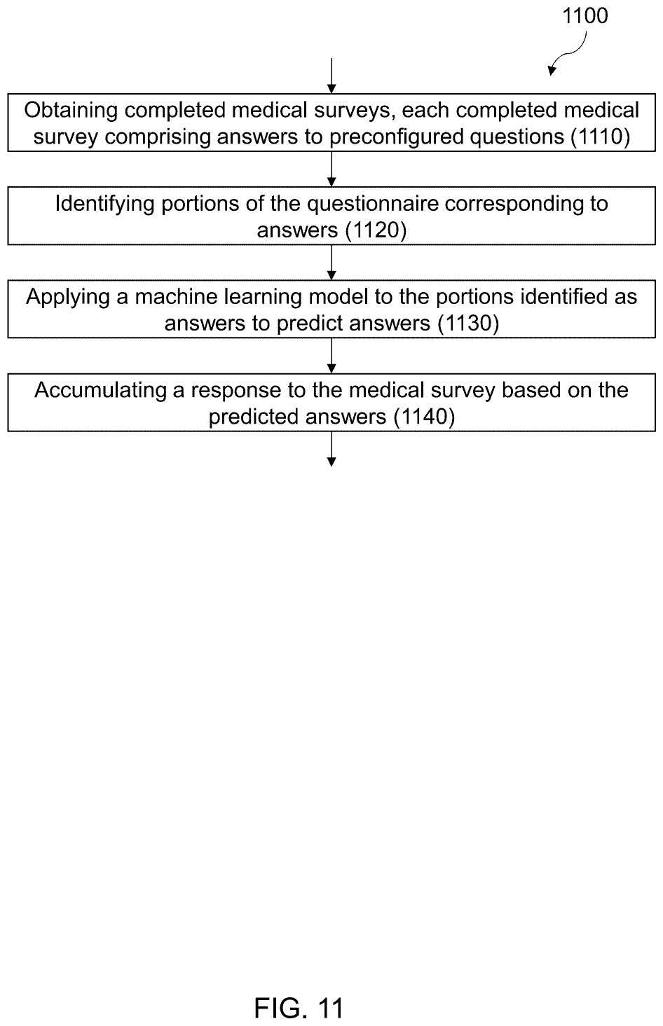

FIG. 11 shows a flow chart of an example computer-implemented method 1100 of extracting clinical patient information from medical surveys. The method 1100 comprises the steps of obtaining 1110 completed medical surveys, each completed medical survey comprising answers to preconfigured questions; identifying 1120 portions of each completed medical survey corresponding to answers; applying 1130 a machine learning model to the portions identified as answers to predict answers; and accumulating 1140 a response to the medical survey based on the predicted answers.

Identifying 1120 portions as corresponding to answers may include making a prediction that the portion correspond to answers.

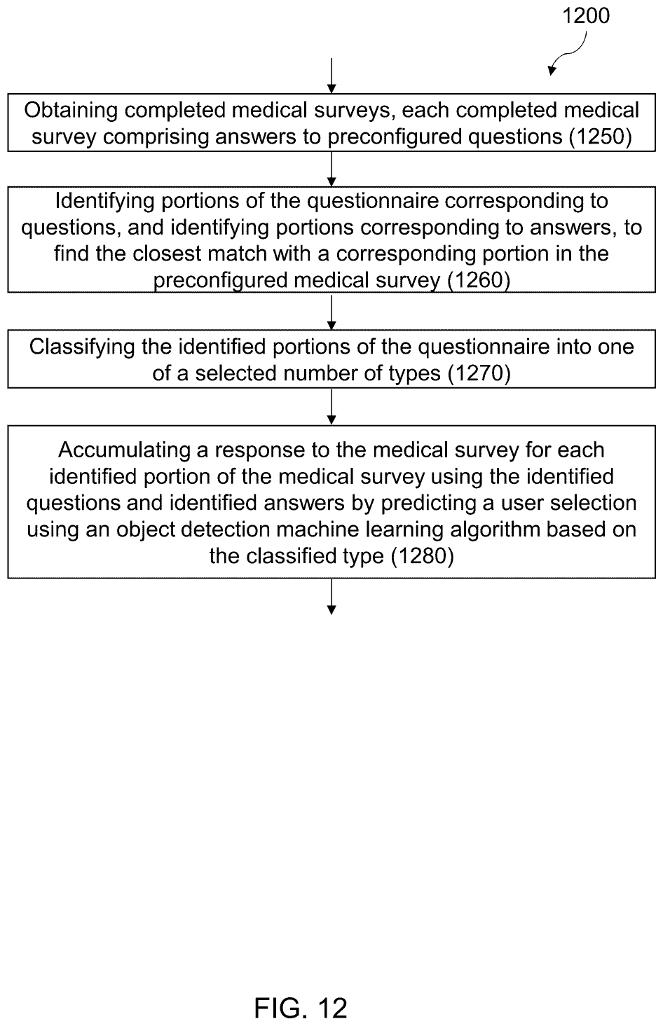

FIG. 12 shows a flow chart of another example computer-implemented method 1200 of extracting clinical patient information from medical surveys. The method 1200 comprises the steps of obtaining 1250 completed medical surveys, each completed medical survey comprising answers to preconfigured questions; identifying 1260 portions of the medical survey corresponding to questions, and identifying portions corresponding to answers, to find the closest match with a corresponding portion in the preconfigured medical survey; classifying 1270 the identified portions of the medical survey into one of a selected number of types; and accumulating 1280 a response to the medical survey for each identified portion of the medical survey using the identified questions and identified answers by predicting a user selection using an object detection machine learning algorithm based on the classified type.

Identifying 1260 portions of the medical survey corresponding to questions, and identifying portions corresponding to answers, to find the closest match with a corresponding portion in the preconfigured medical survey may correspond to step 903 of FIG. 9. Classifying 1270 the identified portions of the medical survey into one of a selected number of types may correspond to the step 905 of FIG. 9.

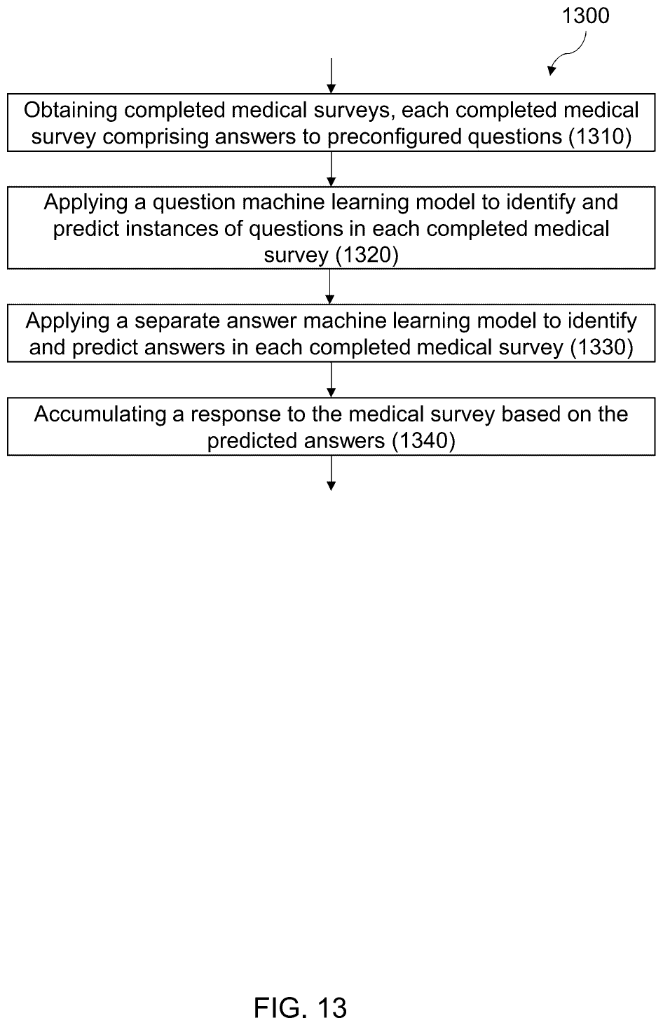

FIG. 13 shows a flow chart of another example computer-implemented method 1300 of extracting clinical patient information from medical surveys. The method 1300 comprises the steps of obtaining 1310 completed medical surveys, each completed medical survey comprising answers to preconfigured questions; applying 1320 a question machine learning model to identify and predict instances of questions in each completed medical survey; applying 1330 a separate answer machine learning model to identify and predict answers in each completed medical survey; and accumulating 1340 a response to the medical survey based on the predicted answers.

This method may correspond to that shown in FIG. 10, where the step of obtaining 1310 completed medical surveys corresponding to steps 1001 and 1003 of FIG. 10; the step of applying 1320 a question machine learning model to identify and predict instances of questions in each completed medical survey corresponds to steps 1005A and 1005B of FIG. 10; and the step of applying 1330 a separate answer machine learning model to identify and predict answers in each completed medical survey corresponds to steps 1007A and 1007B of FIG. 10.

FIG. 14 shows a process flow chart for the steps 1400 performed by a graphical user interface for displaying the result of medical surveys. The process 1400 comprises the steps of: on a first page, prompting 1410 a user to upload a blank medical survey; on a subsequent page, providing 1420 an indication to the user of each identified question and answer section of the blank medical survey along with a question and answer type, and prompting the user to correct any misidentified areas or question and answer types; and on a subsequent page, providing 1430 an indication to the user of the answers to each identified question and answer section for each completed survey based on the identified question and answer type for that identified question and answer section, along with an indication of the confidence level of the indication for each completed survey and answer section for each completed questionnaire.

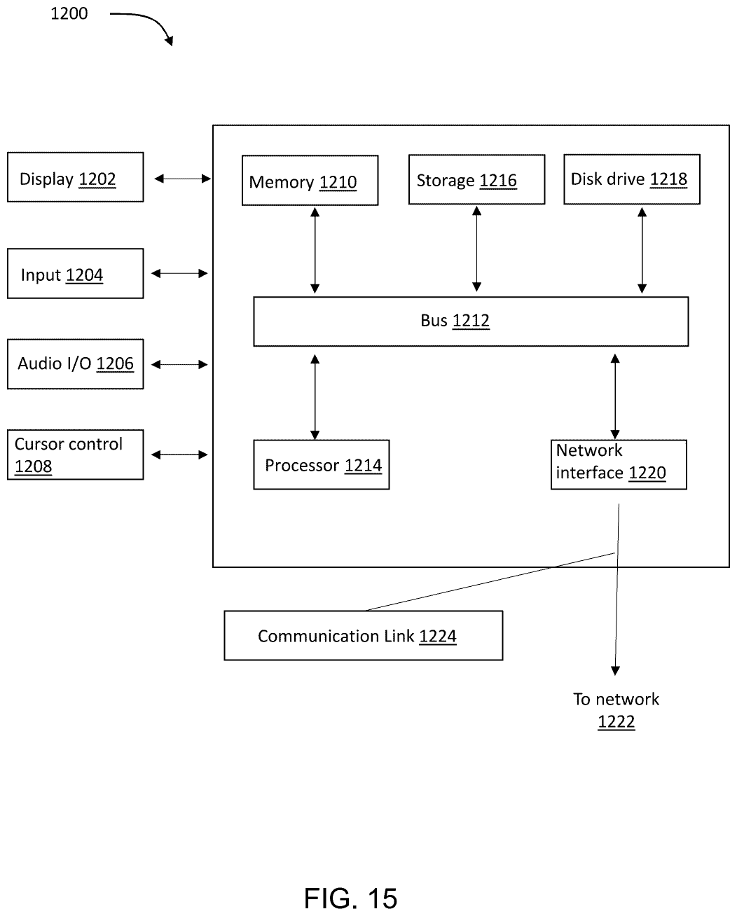

FIG. 15 is a block diagram of a computer system 1200 suitable for implementing one or more embodiments of the present disclosure, including for example any of the methods described above with reference to FIGS. 1 to 14.

The computer system 1200 includes a bus 1212 or other communication mechanism for communicating information data, signals, and information between various components of the computer system 1200. The components include an input/output (I/O) component 1204 that processes a user (i.e., sender, recipient, service provider) action, such as selecting keys from a keypad/keyboard, selecting one or more buttons or links, etc., and sends a corresponding signal to the bus 1212. It may also include a camera for obtaining image data. The I/O component 1204 may also include an output component, such as a display 1202 and a cursor control 1208 (such as a keyboard, keypad, mouse, etc.). The display 1202 may be configured to present a login page for logging into a user account, and is configured to display a user interface such as the user interface described above with reference to FIGS. 1 to 8 and 14. An optional audio input/output component 1206 may also be included to allow a user to use voice for inputting information by converting audio signals. The audio I/O component 1206 may allow the user to hear audio. A transceiver or network interface 1220 transmits and receives signals between the computer system 1200 and other devices, such as another user device, a merchant server, or a service provider server via network 1222. In one embodiment, the transmission is wireless, although other transmission mediums and methods may also be suitable. A processor 1214, which can be a micro-controller, digital signal processor (DSP), or other processing component, processes these various signals, such as for display on the computer system 1200 or transmission to other devices via a communication link 1224. The processor 1214 may also control transmission of information, such as cookies or IP addresses, to other devices.

The components of the computer system 1200 also include a system memory component 1210 (e.g., RAM), a static storage component 1216 (e.g., ROM), and/or a disk drive 1218 (e.g., a solid-state drive, a hard drive). The computer system 1200 performs specific operations by the processor 1214 and other components by executing one or more sequences of instructions contained in the system memory component 1210. For example, the processor 1214 can run the machine learning model(s) described above, and/or the computer-implemented method described above, for example, with reference to FIGS. 12 and 13.

It will also be understood that the modules may be implemented in software or hardware, for example as dedicated circuitry. For example, the modules may be implemented as part of a computer system. The computer system may include a bus or other communication mechanism for communicating information data, signals, and information between various components of the computer system. The components may include an input/output (I/O) component that processes a user (i.e., sender, recipient, service provider) action, such as selecting keys from a keypad/keyboard, selecting one or more buttons or links, etc., and sends a corresponding signal to the bus. The I/O component may also include an output component, such as a display and a cursor control (such as a keyboard, keypad, mouse, etc.). A transceiver or network interface may transmit and receive signals between the computer system and other devices, such as another user device, a merchant server, or a service provider server via a network. In one embodiment, the transmission is wireless, although other transmission mediums and methods may also be suitable. A processor, which can be a micro-controller, digital signal processor (DSP), or other processing component, processes these various signals, such as for display on the computer system or transmission to other devices via a communication link. The processor may also control transmission of information, such as cookies or IP addresses, to other devices.

The components of the computer system may also include a system memory component (e.g., RAM), a static storage component (e.g., ROM), and/or a disk drive (e.g., a solid-state drive, a hard drive). The computer system performs specific operations by the processor and other components by executing one or more sequences of instructions contained in the system memory component.

Logic may be encoded in a computer readable medium, which may refer to any medium that participates in providing instructions to a processor for execution. Such a medium may take many forms, including but not limited to, non-volatile media, volatile media, and transmission media. In various implementations, non-volatile media includes optical or magnetic disks, volatile media includes dynamic memory, such as a system memory component, and transmission media includes coaxial cables, copper wire, and fiber optics. In one embodiment, the logic is encoded in non-transitory computer readable medium. In one example, transmission media may take the form of acoustic or light waves, such as those generated during radio wave, optical, and infrared data communications.

Some common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer is adapted to read.

In various embodiments of the present disclosure, execution of instruction sequences to practice the present disclosure may be performed by a computer system. In various other embodiments of the present disclosure, a plurality of computer systems 1200 coupled by a communication link to a network (e.g., such as a LAN, WLAN, PTSN, and/or various other wired or wireless networks, including telecommunications, mobile, and cellular phone networks) may perform instruction sequences to practice the present disclosure in coordination with one another.

It will also be understood that aspects of the present disclosure may be implemented using hardware, software, or combinations of hardware and software. Also, where applicable, the various hardware components and/or software components set forth herein may be combined into composite components comprising software, hardware, and/or both without departing from the spirit of the present disclosure. Where applicable, the various hardware components and/or software components set forth herein may be separated into sub-components comprising software, hardware, or both without departing from the scope of the present disclosure. In addition, where applicable, it is contemplated that software components may be implemented as hardware components and vice-versa.

Software in accordance with the present disclosure, such as program code and/or data, may be stored on one or more computer readable mediums. It is also contemplated that software identified herein may be implemented using one or more general purpose or specific purpose computers and/or computer systems, networked and/or otherwise. Where applicable, the ordering of various steps described herein may be changed, combined into composite steps, and/or separated into sub-steps to provide features described herein.

The various features and steps described herein may be implemented as systems comprising one or more memories storing various information described herein and one or more processors coupled to the one or more memories and a network, wherein the one or more processors are operable to perform steps as described herein, as non-transitory machine-readable medium comprising a plurality of machine-readable instructions which, when executed by one or more processors, are adapted to cause the one or more processors to perform a method comprising steps described herein, and methods performed by one or more devices, such as a hardware processor, user device, server, and other devices described herein.

Reference throughout this specification to “some embodiments,” or “an embodiment,” means that a particular feature, structure, or characteristic described in connection with the embodiment is included in at least one embodiment. Thus, the appearances of the phrase “in some embodiment,” or “in an embodiment,” in various places throughout this specification are not necessarily all referring to the same embodiment. Furthermore, the particular features, structures, or characteristics may be combined in any suitable manner in one or more embodiments.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

As utilized herein, terms “component,” “system,” “interface,” “unit” and the like are intended to refer to a computer-related entity, hardware, software (e.g., in execution), and/or firmware. For example, a component can be a processor, a process running on a processor, an object, an executable, a program, a storage device, and/or a computer. By way of illustration, an application running on a server and the server can be a component. One or more components can reside within a process, and a component can be localized on one computer and/or distributed between two or more computers.

Further, these components can execute from various computer readable media having various data structures stored thereon. The components can communicate via local and/or remote processes such as in accordance with a signal having one or more data packets (e.g., data from one component interacting with another component in a local system, distributed system, and/or across a network, e.g., the Internet, a local area network, a wide area network, etc. with other systems via the signal).

As another example, a component can be an apparatus with specific functionality provided by mechanical parts operated by electric or electronic circuitry; the electric or electronic circuitry can be operated by a software application or a firmware application executed by one or more processors; the one or more processors can be internal or external to the apparatus and can execute at least a part of the software or firmware application. As yet another example, a component can be an apparatus that provides specific functionality through electronic components without mechanical parts; the electronic components can include one or more processors therein to execute software and/or firmware that confer(s), at least in part, the functionality of the electronic components. In some cases, a component can emulate an electronic component via a virtual machine, e.g., within a cloud computing system.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

It will be appreciated from the discussion above that the embodiments shown in the Figures are merely exemplary, and include features which may be generalised, removed or replaced as described herein and as set out in the claims. In the context of the present disclosure other examples and variations of the apparatus and methods described herein will be apparent to a person of skill in the art.

1. A computer implemented method of extracting data from surveys, the method comprising:
obtaining completed surveys, each completed survey comprising answers to preconfigured questions;
identifying portions of each completed survey corresponding to answers;
applying a machine learning model to the portions identified as answers to predict answers; and
accumulating a response to the survey based on the predicted answers.

2. The computer implemented method of claim 1, wherein accumulating a response to the survey based on the identified answers comprises matching the identified answers to the preconfigured questions.

3. The computer implemented method of claim 1, further comprising:
outputting a display of the answers to each question in a graphical user interface to the user, along with an indication of a location of the answer in the survey and a confidence level that the answer has been correctly predicted by a second machine learning model.

4. The computer implemented method of claim 1, further comprising:
classifying sections of the survey into one of a selected number of different types;
wherein identifying portions of the survey corresponding to answers comprises identifying a portion corresponding to answers for each classified section of the survey.

5. The computer implemented method of claim 4, wherein the selected number of different types comprise: option, table, scale, image, and text.

6. The computer implemented method of claim 4, wherein applying a machine learning model to the portions identified as answers to predict answers comprises applying a machine learning model to the portions identified as answers to predict answers for each classified section based on the classified type of the section.

7. The computer implemented method of claim 1, wherein obtaining completed surveys comprises obtaining an image of completed surveys.

8. The computer implemented method of claim 1, further comprising obtaining from a user an indication from a list of preconfigured surveys of the survey being obtained.

9. The computer implemented method of claim 1, further comprising determining whether each of the surveys belongs to a list of preconfigured surveys; and
in the event that the survey is determined to belong to the list of preconfigured surveys, suggesting to the user a recommended survey to select from the list of preconfigured surveys; and
in the event that the survey does not belong to the list of preconfigured surveys, prompting the user to provide a blank copy of the survey.

10. The computer implemented method of claim 1, further comprising identifying portions of the survey corresponding to questions.

11. The computer implemented method of claim 9, wherein identifying portions of the survey corresponding to questions comprises applying (i) a layout machine learning model configured to split an image of the completed survey into portions that are questions, and (ii) an extract machine learning model configured to extract a question from each portion.

12. The computer implemented method of claim 1, wherein identifying portions of the survey corresponding to answers comprises applying (i) a layout machine learning model configured to split the image into portions that are answers, and (ii) an extract machine learning model configured to extract an answer from each portion.

13. The computer implemented method of claim 1, wherein identifying portions of the survey corresponding to answers comprises generating boxes with size ratio and variable size around portions of the survey and then matching the identified portions to preconfigured portions of a preconfigured survey.

14. A computer-implemented method of extracting data from surveys, the method comprising:
obtaining completed surveys, each completed survey comprising answers to preconfigured questions;
applying a question machine learning model to identify and predict instances of questions in each completed survey;
applying a separate answer machine learning model to identify and predict answers in each completed survey; and
accumulating a response to the survey based on the predicted answers.

15. The computer implemented method of claim 14, wherein obtaining completed surveys comprises obtaining an image of each completed survey.

16. The computer implemented method of claim 14, wherein applying a question machine learning model to identify and predict instances of questions in each completed survey comprises the steps of (i) splitting the image into portions that are questions, and (ii) extracting a question from each portion.

17. The computer implemented method of claim 16, wherein splitting the image into portions that are questions comprises identifying portions by generating boxes with size ratio and variable size around portions of the survey and then matching the identified portions to preconfigured portions of a preconfigured survey.

18. The computer implemented method of claim 15, wherein applying an answer machine learning model to identify and predict answers in each completed survey comprises the steps of (i) splitting the image into portions that are answers, and (ii) extracting an answer from each portion.

19. The computer implemented method of claim 18, wherein splitting the image into portions that are answers comprises identifying portions by generating boxes with size ratio and variable size around portions of the survey and then matching the identified portions to preconfigured portions of a preconfigured survey.

20. A graphical user interface configured to display results of surveys, the graphical user interface configured to:
on a first page, prompt a user to upload a blank survey;
on a subsequent page, provide an indication to the user of each identified question and answer section of the blank survey along with a question and answer type, and prompting the user to correct any mis-identified areas or question and answer types; and
on a subsequent page, provide an indication to the user of the answers to each identified question and answer section for each completed survey based on the identified question and answer type for that identified question and answer section, along with an indication of a confidence level of the indication for each completed survey and answer section for each completed survey.

21. A computing device comprising a display screen, the computing device being configured to display on the screen:
a prompt to a user to upload a blank survey;
after the user has uploaded a blank survey, an indication to the user of each identified question and answer section of the blank survey along with a question and answer type, and prompting the user to correct any mis-identified areas or question and answer types; and
subsequently, an indication to the user of the answers to each identified question and answer section for each completed survey based on the identified question and answer type for that identified question and answer section, along with an indication of a confidence level of the indication for each completed survey and answer section for each completed survey.
